(19) United States
(12) Patent Application Publication
(10) Pub. No.: US 2021/0369806 A1
(43) Pub. Date: Dec. 2, 2021

(54) HERBAL MIXTURE FOR VIRAL DISEASE SYMPTOMS

(71) Applicant: Ganiyu Ayinla Jaiyeola, Grand Rapids, MI (US)

(72) Inventor: Ganiyu Ayinla Jaiyeola, Grand Rapids, MI (US)

(21) Appl. No.: 17/302,142

(22) Filed: Apr. 26, 2021

Related U.S. Application Data
(60) Provisional application No. 63/030,574, filed on May 27, 2020.

(51) Int. Cl. A61K 36/9068; A61M 11/04; A61K 9/00; A61K 36/9066; A61K 36/88; A61K 36/752; A61K 36/38; A61K 35/644; A23L 2/52; A23L 33/105

(52) U.S. Cl. CPC A61K 36/9068; A61M 11/04; A61K 9/0043; A61K 9/0095; A61K 36/9066; A23V 2002/00; A61K 36/752; A61K 36/38; A61K 35/644; A23L 2/52; A23L 33/105; A61K 36/88

(57) ABSTRACT

This invention pertains to an herbal paste comprising a mixture comprising 15 to 25 parts by weight ginger root, 15 to 25 parts by weight turmeric root, 15 to 25 parts by weight garlic 15 to 25 parts by weight lime fruit, 15 to 25 parts by weight lemon fruit, 0.5 to 1.5 parts by weight bitter kola nut, 15 to 25 parts by weight natural honey and B. optionally up to 50 parts water per 100 parts of Mixture A. Also claimed is a drink from the herbal mixture and a method of using the herbal mixture for active inhalation.

This application claims the benefit of U.S. Provisional Application No. 63/030,574 filed May 27, 2020, and incorporated herein by reference

### BACKGROUND OF THE INVENTION

Various herbs have been known for ages to help with cough, fever, respiratory, and other symptoms. An outbreak of Coronavirus disease in 2019 has created a pandemic that shows various symptoms of cough, fever, and respiratory symptoms. This invention created an herbal mixture that relieves the symptoms created by the Coronavirus disease as well as other diseases that produce coughs and other respiratory symptoms.

### BRIEF SUMMARY OF THE INVENTION

This invention is an herbal mixture for relieving symptoms arising from cough and respiratory symptoms, in particular those caused by COVID-19 virus also known as SARS-CoV-2. The symptoms that are being caused by COVID-19 include cough, shortness of breath or difficulty in breathing, fever, chills, muscle pain, sore throat, loss of taste or smell, and gastrointestinal symptoms like nausea, vomiting, or diarrhea. The invention pertains to a mixture of ginger root, turmeric, garlic, lime fruit, lemon fruit, natural honey, and bitter kola nut.

### DETAILED DESCRIPTION OF THE INVENTION

The instant invention comprises of an herbal paste that is made with ginger root, turmeric, garlic, lime fruit, lemon fruit, natural honey, and bitter kola nut. Typically, the herbal paste comprises

A. A Mixture comprising:

15 to 25 parts by weight Ginger root

15 to 25 parts by weight Turmeric

15 to 25 parts by weight Garlic

15 to 25 parts by weight Lime Fruit

15 to 25 parts by weight Lemon Fruit

0.5 to 1.5 parts by weight Bitter Kola Nut

15 to 25 parts by weight Natural Honey and

B. optionally up to 50 parts water per 100 parts of Mixture A.

More typically the herbal paste comprises:

A: A Mixture comprising:

20 parts by weight Ginger Root

20 parts by weight Turmeric Root

20 parts by weight Garlic

20 parts by weight Lime Fruit

20 parts by weight Lemon Fruit

1 part by weight Bitter Kola Nut

20 parts by weight Natural Honey and

B: optionally up to 50 parts water per 100 parts of the Mixture A.

### Ginger Root

Ginger is a plant with leafy stems and yellowish green flowers. Ginger is native to warmer parts of Asia, such as China, Japan, and India, but now is grown in parts of South America and Africa. It is also now grown in the Middle East to use as medicine and with food. Depending on the variety, the inside of the root can be yellow, red, or white. It's harvested by pulling the entire plant out of the soil, removing the leaves and cleaning the root. Ginger is available fresh, dried, or made into tablets, capsules, and liquid extracts. For purposes of this invention, it is preferred to use the fresh root. The fresh root is readily available in most markets. If fresh root is not available, dried ginger may be used in its place. A liquid extract of ginger root may be used although it is not preferred. Fresh ginger can last in the refrigerator for up to three weeks as long as you don't peel it. Fresh ginger can also be frozen if you don't plan to use it right away. When using fresh ginger root, it is preferred to peel it before using it. To make it easier to blend, the ginger root may be chopped into smaller pieces when added into Mixture A. Ginger Root is used in the herbal paste at 15 to 25 parts by weight, typically 20 parts by weight.

### Turmeric Root

Turmeric is an herb plant that grows in India and Central America. Like ginger the root is harvested and is readily found in most markets. You can also buy turmeric root dried and ground as a spice. It is preferable to use the fresh turmeric root in the instant invention, however if the root is not available one can use the dried and ground spice form. When using fresh turmeric root, it is preferred to peel it before using it. To make it easier to blend, the turmeric root may be chopped into smaller pieces when added into Mixture A. Turmeric root is used in the herbal paste at 15 to 25 parts by weight typically 20 parts by weight.

Garlic

Garlic is a species in the onion genus, Allium. Although the leaves and flowers of the plant are also edible, the bulb, comprised of 10-20 cloves, is most frequently eaten. It's covered in a paper-like husk that's typically removed before consumption. For purposes of this invention, the cloves are to be used to create the herbal paste. Garlic is readily available at the supermarket typically in the produce section along with the onions and potatoes. It is sold individually as full heads. Garlic is also sold in jars with olive oil, either as whole, peeled cloves or minced cloves. You will also find it in different forms, such as freeze-dried and garlic powder. Fresh garlic is preferred for the instant invention. If whole heads are purchased, the paper-like husk needs to be removed and the cloves obtained for use. Less desirable is jarred garlic because it may be contaminated with substances such as olive oil. Freeze dried and powdered forms of garlic may be used when fresh garlic is not available. The garlic cloves may be chopped prior to adding into Mixture A to make it easier for blending. Garlic is used in the herbal paste at 15 to 25 parts by weight typically 20 parts by weight.

### Lime Fruit

Lime Fruit, is a citrus fruit, which is typically round, green in color, 3-6 centimeters in diameter, and contains acidic juice vesicles. For purposes of this invention, it is preferred to use the whole lime fruit. The lime fruit may be added whole, sliced, chopped or otherwise mashed before adding to the Mixture. Alternatively, the juice may be removed from the lime fruit and added to the mixture and then the remaining body of the lime fruit may be added. Alternatively, one can zest the lime fruit adding the zest and then add the reminder of the lime to the mixture. Alternatively, one can zest the lime, remove the juice and then add all of the components of the lime fruit to the mixture. One skilled in the art can determine the best way to process the lime fruit to incorporate it into the Mixture to produce the Herbal Paste. Lime fruit is used in the herbal paste at 15 to 25 parts by weight typically 20 parts by weight.

### Lemon Fruit

A lemon is a bright yellow citrus fruit that is known for its acidic juice. The yellow, oval-shaped citrus fruit grows on flowering evergreen trees and is cultivated commercially and in backyard gardens around the world. Lemons thrive in mild, warm climates like Mexico, California, and India. For purposes of this invention, it is preferred to use the whole lemon fruit. The lemon fruit may be added whole, sliced, chopped or otherwise mashed before adding to the Mixture. Alternatively, the juice may be removed from the lemon fruit and added to the mixture and then the remaining body of the lemon fruit may be added. Alternatively, one can zest the lemon fruit adding the zest and then add the reminder of the lemon fruit to the mixture. Alternatively, one can zest the lemon fruit, remove the juice and then add all of the components of the lemon fruit to the mixture. One skilled in the art can determine the best way to process the lime fruit to incorporate it into the Mixture to produce the Herbal Paste. Lemon fruit is used in the herbal paste at 15 to 25 parts by weight typically 20 parts by weight.

### Bitter Kola Nut

The bitter kola nut is the fruit of the kola tree (Cola acuminata and Cola nitida), indigenous to West Africa. The trees, which reach heights of 40 to 60 feet, produce a star-shaped fruit. Each fruit contains between two and five bitter kola nuts. The bitter kola nuts may be placed directly into the Mixture A or they may be ground before placing into the Mixture A. Bitter Kola Nut is used in the herbal paste at 0.5 to 1.5 parts by weight, typically 1 part by weight.

### Natural Honey

Honey is a sweet, syrupy, golden-colored liquid made by honeybees. Honeybees store honey in the beehive to use for food and nutrients. Natural honey comes directly from the hive and does not undergo any processing or pasteurization before bottling. Natural honey does not have any added sugars or preservatives. The natural honey may be warmed slightly to help it liquify it and make it easier to blend into Mixture A. One skilled in the art will know how much to heat it to liquify it. Natural honey is used in the herbal paste at 15 to 25 parts by weight typically 20 parts by weight.

### Water

Water is optionally added to mixture A during the blending step to allow them to combine together easier. Water may be added to help produce the appropriate consistency. The amount of water added should be no more than 50 parts by weight per 100 parts by weight of the mixture A. Typically, no more than 40 parts by weight of water per 100 parts by weight of the herbal paste, more typically no more than 25 parts by weight of water per 100 parts by weight of the mixture A. The water may be drizzle into the mixture A or it may be added in its entirety. One skilled in the art will know how to add the water to the paste to reach the optimal consistency for the herbal paste. Potable water should be used such as tap water, distilled water, filtered water, bottled, and others.

Additional Ingredients:

Additional ingredients may be added to the paste depending on whether the paste is going to be used as a beverage or for active inhalation. Examples of additional ingredients include other fruits such as Apples, Pears, Oranges, spices such as Thyme, Nutmeg and others known to those skilled in the art, essential oils such as peppermint, mint, lavender, eucalyptus, geranium, cypress, bergamot and others known to one skilled in the art.

In preparing the herbal paste the ginger root, turmeric, garlic, lime fruit, lemon fruit, and bitter kola nut (herein after referred to as Herbal Ingredients) should be washed to remove surface contaminants. Typically, the Herbal Ingredients are washed with tap water. Other types of water may be used to wash the Herbal Ingredients such as bottled water, boiled water typically cooled so not to cook the herbal ingredients, distilled water, filtered water, etc. One skilled in the art will be able to adequately select the appropriate type of water to clean the Herbal Ingredients.

The Herbal Paste is produced by processing together the Herbal Ingredients (ginger root, turmeric root, garlic, lime fruit, lemon fruit, and bitter kola nut) to produce a rough or fine puree.

If small quantity of the Herbal Ingredients is desired, processing can take place by hand using various devices suitable for blending, mashing or grinding typically found in kitchen households. If a larger quantity of the Herbal Ingredients is desired, one skilled in the art would use mechanical processing devices such as those found in the typical house hold such as blenders, mixer, food processors to commercial blenders or food processors. It is desired to reduce the Herbal Ingredients to a rough to fine puree during this process and allow them to be combined with the honey. If the Herbal Ingredients appear dry, optional water may be added to help reduce them to the rough or fine puree. Once the Herbal Ingredients have been processed the natural honey is then added and they are all blended together to form the Herbal Paste of this invention.

The herbal paste may then be placed in a container, and covered to keep out contaminants. It is preferred to keep the paste at room temperature (between 68° and 74° F.). For best effects it is preferred to use the herbal paste within 1 to 2 hours after producing it. However, if one is not going to use all of the paste in the immediate future one may place the paste in a refrigerated storage environment of 35° to 50° F., more preferably, 38° to 42° F. Typically, the paste should be used within 1 to 2 days of producing but no more that 5 to 7 days after using when refrigerated. One skilled in the art will be able to tell when the old herbal paste should be discarded.

The Herbal Paste is used to relieve symptoms associated with cough, fever, respiratory, and other symptoms (hereinafter “Symptoms”), in particular those association with COVID-19. One way to use the Herbal Paste to relieve these symptoms is to produce a beverage from the Herbal Paste. The beverage comprises 1 part of potable water to 1 part of Herbal Paste. The beverage is produced by adding the potable water to the paste in a container and mixing the water and paste together. The water should be heated prior to adding to the paste, typically between 130 and 160° F. The container used for holding the beverage can be a typical drinking cup that can hold hot beverages such as a tea cup or coffee cup. It is preferred that the beverage container not have a cover so that the vapors from the beverage are being released. Once the beverage has been formed the beverage should be consumed slowly while warm, typically between 130 and 150° F. While consuming the beverage the user should breathe normal to allow for passive vapor inhalation to occur. The passive vapor inhalation occurs into the lungs through the nose as the beverage is consumed from the container. After the beverage has been consumed there may be residual Herbal Paste left in the container. It is recommended to also consume this residual Herbal Paste that is left in the container. It is recommended to consume an Herbal Paste beverage once a day while experiencing the symptoms.

Another way to use the Herbal Paste to relieve these symptoms is by active vapor inhalation of the Herbal Paste. In order to carry out active vapor inhalation, a mixture of 1-part potable water and 1-part Herbal paste are combined together in a bowl of approximately 8 to 12 inches in diameter. The potable water should be warmed, typically to a temperature of 180 and 200° F. before combining it with the Herbal Paste. The Herbal Paste and water are then mixed together typically with plastic, silicone or wooden utensil. One skilled in the art will be able to determine the appropriate utensil for mixing together the Herbal paste and potable water. In order to carry out the active vapor inhalation the head is place over the bowl approximately 5 to 7 inches above the surface of the bowl. The eyes should be shut to prevent the vapor from contacting the eyes. In order to allow sufficient vapor to be inhaled the head and bowl should be covered with a cloth such as a terry cloth. The cloth should allow some of the vapor to escape and fresh air to enter so as not to suffocate the user. Once the head is placed above the bowl and the head and bowl area is covered the user should inhale the vapor by breathing normally through their nose for a period of 1 to 5 minutes typically 1 to 3 minutes. If the user has any difficulty with breathing, they should stop the procedure. It is recommended to carry out the active vapor inhalation at least once a day while experiencing the symptoms.

1. An herbal paste comprising:
A. A mixture comprising:
15 to 25 parts by weight ginger root
15 to 25 parts by weight turmeric root
15 to 25 parts by weight garlic
15 to 25 parts by weight lime Fruit
15 to 25 parts by weight lemon Fruit
0.5 to 1.5 parts by weight bitter kola nut
15 to 25 parts by weight natural honey and
B. optionally up to 50 parts water per 100 parts of Mixture A.

2. The herbal paste as claimed in claim 1 wherein there is
20 parts by weight ginger root
20 parts by weight turmeric root
20 parts by weight garlic
20 parts by weight lime fruit
20 parts by weight lemon fruit
1 part by weight bitter kola nut
20 parts by weight natural honey
in Mixture A.

3. A beverage comprising 1 part of the herbal paste as claimed in claim 1 and 1 part of potable water wherein the beverage is warmed to a temperature of between 130° and 160° F.

4. A method of active inhalation of an herbal paste wherein the method comprises
i. producing a mixture of one part herbal paste as claimed in claim 1 and one part of potable water that has been heated to a temperature of 180° and 200° F. into an open top container to produce a liquid giving off vapors.
ii. placing your head with your eyes closed over the container at a sufficient distance above the liquid but within the space of the vapors.
iii. covering your head with a cloth to enclose your head and container.
iv. breathing in the vapors for a sufficient period of time through the nasal passages.
